(19) United States
(12) Patent Application Publication
(10) Pub. No.: US 2025/0275688 A1
(43) Pub. Date: Sep. 4, 2025

(54) A DEVICE AND A METHOD FOR SENSING VIBRATIONS IN A HUMAN'S SKIN EMANATING FROM BLOOD FLOW BELOW THE SKIN

(71) Applicants: AALBORG UNIVERSITET, Aalborg Øst (DK); REGION NORDJYLLAND, Aalborg Øst (DK)

(72) Inventors: Samuel Emil SCHMIDT, Aalborg Øst (DK); Oliver Thomsen DAMSGAARD, Aalborg Øst (DK); Jeppe Hagstrup CHRISTENSEN, Aalborg Øst (DK)

(21) Appl. No.: 18/861,641

(22) PCT Filed: May 3, 2023
(86) PCT No.: PCT/EP2023/061618

§ 371 Date: Oct. 30, 2024

(30) Foreign Application Priority Data
May 3, 2022 (DK) PA202270233

(51) Int. Cl. A61B 5/11; A61B 5/00; A61B 5/0295

(52) U.S. Cl. CPC A61B 5/1102; A61B 5/0295; A61B 5/6833

(57) ABSTRACT

The present invention relates to a device (1) and a method for sensing vibrations in a human's skin (2) emanating from blood flowing in a natural or artificial blood vessel or an arteriovenous fistula (AVF) (3) located below the skin. The device comprises a sensor (4), a controller (5), a transmitter (6), and an electrical power source (7). In particular, the invention relates to such a device that is configured to be fastened to the human and carried around by the human during use of the device. The measured signals are analysed, and if the blood flow is below a pre-selected blood flow threshold, an alarm signal is issued to warn e.g. a responsible medical doctor that there is a need for further assessment.

### FIELD OF THE INVENTION

The present invention relates to a device and a method for sensing vibrations in a human's skin emanating from blood flowing in a natural or artificial blood vessel or an arteriovenous fistula (AVF) located below the skin. In particular, it relates to such a device that is configured to be fastened to the human and carried around by the human during use of the device. The measured signals are analysed, and if the blood flow is below a blood flow threshold, an alarm signal is issued to warn e.g. a responsible medical doctor that there is a need for further assessment.

### BACKGROUND OF THE INVENTION

An arteriovenous fistula (AVF) is the artificial connection of an artery and a vein, creating a shortcut for the blood flow. It is used when a patient is to receive haemodialysis and greatly increases the blood pressure and flow of blood in the vein. The result is a “matured” vein, where the wall of the vein becomes thicker and more robust which is necessary for the patient to be able to receive haemodialysis 3-4 times a week. An AVF can also be referred to as a shunt.

Dysfunction of an AVF is frequent, and often stenosis or formation of blood clots are observed. If these problems are not recognized and dealt with within 24 hours, the AVF is chronically damaged. If stenosis happens or a blood clot forms in the AVF, the blood flow will change, resulting in a change in the turbulence created by the blood flow. When listening to the AVF with a stethoscope, the turbulence sounds characteristically like a spilling wave. In order to check the correct functioning of an AVF, the sound is typically to be checked with a stethoscope at regular intervals. When a patient having an AVF is not in hospital, these checks are typically performed by the patient him-or herself. However, these patients are often elderly people with other diseases, and many have decreased hearing abilities, or they are not fully aware of the need to perform the necessary checks at regular intervals. Furthermore, it may be found inconvenient to perform such checks as often as needed. Therefore, there is a high risk that a possible dysfunction of an AVF is not registered early enough to take appropriate action.

Thus, there is a need for an improved surveillance system and method for monitoring that an AVF is functioning as intended.

### OBJECT OF THE INVENTION

It is an object of the present invention to provide a device and a method with which it is possible to monitor the functioning of an AVF.

It is another object of the present invention to provide such a device which can be fastened to a patient's body, such as to an arm, and carried around while the monitoring takes place.

It is an object of at least some embodiments of the invention to provide a device and a method which can automatically issue a warning if the AVF is not functioning as intended.

It is a further object of the present invention to provide an alternative to the prior art.

In particular, it may be seen as an object of the present invention to provide a device and a method that solves the above mentioned problems of the prior art.

### SUMMARY OF THE INVENTION

Thus, the above described object and several other objects are intended to be obtained in a first aspect of the invention by providing a device for sensing vibrations in a human's skin emanating from blood flowing in a natural or artificial blood vessel or an arteriovenous fistula (AVF) located below the skin, the device comprising:
a sensor configured to:
   sense the vibrations when placed either directly or indirectly in contact with the human's skin in close proximity to the natural or artificial blood vessel or AVF, and
   provide a sensor readout representing said vibrations;
a controller configured to sample the sensor readout and provide a sampled readout,.
a transmitter configured to transmit the sampled readout to an external processing unit, and
an electrical power source, such as a battery, for supplying electrical power to the sensor, controller, and transmitter,
   wherein the device is configured to be fastened to the human and carried around by the human during use of the device.

In the following, the terms “human”, “person” and “patient” can be used interchangeably.

Such a device can be used in a system according to the third aspect of the invention as will be described below. The system can e.g. be used to monitor the condition of a newly surgically constructed AVF on a patient before beginning haemodialysis treatment. Hereby it can be ensured that the AVF is in the correct condition and well-functioning so that the treatment can be carried out in a satisfactory and safe manner. Another possible use could be for monitoring of after peripheral artery bypass surgery, such as Femoral Popliteal Bypass or femoral-tibial bypass.

The vibrations being measured are due to the turbulence in the blood flow in the natural or artificial blood vessel or in the AVF. Such vibrations give rise to a sound which can be observed by a doctor by use of a stethoscope. These are also the vibrations to be measured by a device according to the present invention.

By “in close proximity” to the natural or artificial blood vessel or AVF is preferably meant within 5-10 centimetres. The device should preferably not to be placed directly on top of the cicatrice of an AVF operation or on top of the medical patch or plaster covering the cicatrice. Furthermore, it should not be too far away from the location of origin of the vibrations.

When the sampled readout has been sent to the external processing unit, it can be used for different types of analysis and conclusions based thereon. It can e.g. be used for determining whether or not there is sufficient blood flow in an AVF and thereby to monitor whether an inserted AVF keeps functioning as intended. More detailed descriptions of the use of the device will be given below.

The transmitter may be controlled by the controller.

In embodiments wherein the power source is a battery, it should preferably have a battery life corresponding at least to the intended measurement period. The lifetime can be extended by the device being configured to enter into a low-power/shelf mode where minimal energy is required. It may be possible to activate the device from low-power/shelf mode by a command sent from an external device or by receipt of sensor input.

The sensor may be a photo plethysmography sensor, an accelerometer, or a sound sensor, such as a microphone. However, the scope of protection covers all types of sensors which are suitable for measuring the types of vibrations mentioned above. When the sensor comprises an accelerometer, it may e.g. be in the form of a LIS2D-type accelerometer. Typically there will only be one sensor in the device, but in principle there could also be more than one of the same type or of different types. By using more than one sensor, it would be possible to measure over a larger area whereby the risk of errors caused by slight misalignments could be minimised. Additional sensors could e.g. be a photo plethysmography sensor, an electrocardiogramaensor, or a pressure sensor.

In some embodiments of the invention, the device is in the form of a patch carrying the sensor, the controller, the transmitter, and optionally the electrical power source. Such a patch may also be referred to as a plaster, and it can be defined as a unit comprising the other components mentioned. It may e.g. be configured to be fastened to the human's skin by adhesive, such as in the same manner as for a plaster used for the protection of a wound. An example of such an embodiment will be shown in the figures. When the device is not in the form of a patch, it may e.g. be fastened by a strap or by a loop-and-hook fastener. In such embodiments it should be ensured that the fastening does not compromise blood flow in the body part, such as an arm, to which it is fastened.

In embodiments wherein the device is in the form of a patch, one or more of the sensor, the controller, the transmitter, and the electrical power source may be embedded in the patch. By “embedded in” is meant that the outer surface of the patch at least partly covers the embedded one or more component(s). Hereby the device can be provided as a unit which can easily be fastened to the human. In such embodiments, the outer surface which at least partly forms a shell may be made from a biodegradable material. Furthermore, the embedded one or more component(s) can be protected e.g. by a water-proof outer layer of the patch or by the shell being made from a water-impermeable material. It may e.g. be made from a biodegradable polyester waterproof and breathable fabric.

It may be advantageous to have the shell produced in two or more sizes to fit arms of different sizes and circumferences.

The hardware electronics of the device may be refurbishable. This may e.g. be possible by the shell encapsulating the hardware being configured so that it can be pried open (by design) to reuse the hardware by placing the hardware with a new battery in a new shell. In some embodiments, the battery is rechargeable and can be reused as well. In some embodiments of the invention, the transmitter is configured to transmit the sampled readout as a radio signal within in the ISM band from 2.402 GHz to 2.48 GHz. The ISM radio bands are portions of the radio spectrum reserved internationally for industrial, scientific and medical purposes, excluding applications in telecommunications. An example of such a method of transmitting data is by use of Bluetooth technology.

The device may further comprise a receiver configured to receive external input signals in response to which the controller is controllable so that the sampling of sensor readout can be controlled via the external input signals. In such embodiments, the transmitter and receiver may be combined as a transceiver component.

The device may further comprise a memory for, at least temporarily, storing the sampled readout. Such a memory may be provided in the controller or it may be a separate component. By using a memory for storage of the sampled readout until it is transmitted to the external processing unit, it will be possible to re-send it in case an error occurs during the transmission. Hereby it can be avoided that the data is lost.

In some embodiments of the invention, the controller is configured to perform predetermined processing of the sensor readout to arrive at the sampled readout. Such processing could e.g. be removal of potential noise, such as 50 Hz noise caused by the electricity network.

The device may be provided with more hardware components than those mentioned above. In some embodiments, the device comprises an internal real-time clock (RTC) used to keep track of the time. Hereby it will e.g. become possible to time the recording and ensure that the device can plan and start a recording without having to receive a control signal from e.g. a smartphone.

In some embodiments, the device comprises a contact microphone, such as a piezo buzzer. Such a contact microphone works two-ways and can therefore be used both for recording and transmitting. This can enable the device to make sound recordings and play simple tunes used for alarms or notifications.

In some embodiments of the invention, the device comprises a dynamic speaker enabling healthcare personnel to speak directly to the patient/user. This option can e.g. be used for trouble shooting in case the patient/user has questions on e.g. error messages or blinking LEDs. It may also be used to provide instructions to the patient/user e.g. to sit down and rest before a measurement is started.

The device may be configured to be in a state of one among several possible modes of operation enabling various functionality. The modes may typically be two or more of the following: shelf/low-power, sleep, awake, boot, connecting, measurement, transmit, command, debug. Low power mode may be used to increase the shelf life; sleep mode may be between recordings; awake mode may be when recording or other functions are activated; software booting mode may be after reset/failure; connecting mode may be when connecting to a gateway via BLE/Wi-Fi; measurement/recording mode may be when the flow in e.g. an AVF is being measured; transmitting mode may be when transmitting data to a gateway via BLE/Wife; command and debug modes may be used for communicating with the sensor via an external device/gateway.

In a second aspect, the invention relates to a computer-implemented method for determining flow characteristics of a blood flow in a natural or artificial blood vessel or an arteriovenous fistula (AVF) located below a human's skin, wherein the method utilizes a device according to the first aspect of the invention, the method comprising the following steps:
providing, by use of the device, sampled readout spanning a pre-selected time interval,
receiving the sampled readout from the transmitter,
processing the sampled readout to determine corresponding flow characteristics of the blood flow in the natural or artificial blood vessel or AVF,
analysing the determined flow characteristics to establish whether or not the blood flow is at or above a blood flow threshold in the natural or artificial blood vessel or AVF, and
   if the blood flow is at or above the blood flow threshold, repeating the above steps after a pre-determined first period of time, and
   if the blood flow is below the blood flow threshold, issuing an alarm signal.

The blood flow threshold may be a pre-determined, such as constant, value. It may also be a value which is determined by a supervised machine-learning process implemented in the method. In such embodiments the threshold typically varies as part of the training of the model; an examples will be described in more details below. The alarm signal may then be issued when the determined flow characteristics is not recognised as “sufficient blood flow”, i.e. corresponding to “blood flow is above the blood flow threshold”.

The situation wherein the blood flow is at or above the blood flow threshold may be referred to as the natural or artificial blood vessel or the AVF being functional, and the opposite situation wherein the blood flow is below the threshold may be referred to as the natural or artificial blood vessel or the AVF being dysfunctional.

The alarm signal may be used in different manners and may vary depending on whether the person on which the measurements are done is at hospital or at home. In both cases, the alarm may be sent to a medical doctor who can then decide what to do and take appropriate action. An advantage of the method is therefore that supervision can be performed from a distance and without the need for the person to perform the control him-or herself. If desired, it may e.g. be possible to build an indicator, such as a LED, into the device which can visually indicate to the person wearing the device whether or not an alarm has been issued. This may increase the feeling of safety to the person who might otherwise feel unsafe by no longer being at the hospital.

In some embodiments of the invention, an “ok” signal is issued when the blood flow is at or above the blood flow threshold. This signal may e.g. be sent to an indicator, such as an LED, so that it can be visibly confirmed that everything is as it should be.

The step of processing the sampled readout may comprise an initial step of assessing the quality of the received sampled readout, and:
if the sampled readout fulfils a pre-selected quality criteria, continue the processing, and
if the sampled readout does not fulfil the pre-selected quality criteria, discontinue the step of processing and wait for a pre-selected second period of time before repeating the step of processing on a subsequently sampled readout.

The step of assessing the quality may comprise determining how many times in a row, if any, it has been determined that the sampled readout does not fulfil the pre-selected quality criteria, and if this has happened more than a pre-selected number of times, the step of analysing is continued on the last sampled readout despite the non-fulfilment of the pre-selected quality criteria. By “if any” is meant to be included that the number may be zero in case it is the first time this happens after the last time the sampled readout did fulfil the pre-selected quality criteria.

The step of analysing the determined flow characteristics may comprise:
assigning a classification to the determined flow characteristic,
comparing the assigned classification to the correspondingly assigned classification of at least one previously analysed flow characteristic, and
based thereon, establishing whether or not the blood flow in the natural or artificial blood vessel or AVF is at or above the blood flow threshold.

In the last mentioned embodiments of a computer-implemented method, the method may further comprise determining how many times in a row, if any, it has been determined that the blood flow is below the blood flow threshold, and
when this has been determined a pre-selected number of times, issuing the alarm signal, and
when this has not been determined the pre-selected number of times, repeating the method steps after the pre-selected second period of time.

The first and second periods of time may be different. Typically, the first time period for when to make a new recording after determining that the blood flow is at or above the blood flow threshold is longer than the second time period, since when there is sufficient blood flow, there is no need to check again quickly. On the other hand, when the blood flow is below the blood flow threshold, it is relevant to check again whether the too low blood flow was just due to a temporary situation and to follow-up on a possible negative development of the condition for the patient being monitored. The first and the second periods of time may be fixed values or they may be possible to adjust over time e.g. depending on the elapse of time since an AVF was inserted.

In a third aspect, the invention relates to a system for determining flow characteristics of a blood flow in natural or artificial a blood vessel or an arteriovenous fistula (AVF) located below a human's skin, the system comprising:
a device according to the first aspect of the invention, and
a processing unit configured to perform a method according to the second aspect of the invention in order to determine the flow characteristics.

The system may further comprise a smartphone or a hub via which the processing unit can communicate with the device. Alternatively, the processing unit may be the smartphone or the hub itself. In some embodiments of the invention, the smartphone or hub can also receive messages from the control unit asking for a new recording before the next planned recording and alarm or inform the patient, if he/she is being recalled to the hospital for further assessment e.g. of their AVF.

The communication may take place via the Internet or via a GSM network. It may be possible to transmit the sampled readout directly to a cloud server so that the smartphone or hub can be omitted provided that the device is configured for such communication.

In some embodiments of a system according to the third aspect of the invention, the processing unit is a cloud server where data is analysed and stored.

Thus, in such embodiments, the system may consist of three parts:
1) the device mounted on the patient and with which the measurements are performed,
2) a gateway hub or smartphone app communicating between the device and a cloud server, and
3) a cloud server where data is analysed and stored.

In the future, technological advancements might open the possibility that the power consumption of electronic components used for wireless data communication has become so low that it is possible for the device to handle communication with the cloud server by itself, and exclude the use of a gateway hub/smartphone app. Alternatively, battery technology might provide batteries with much higher capacities at smaller sizes and lower weight compared to today, so that it would be possible to run wireless communication with the cloud server directly from the device without increasing the size or weight of the device.

As mentioned above, the device or system may be configured to issue an alarm, if the blood flow is below the blood flow threshold. Such an alarm signal may be issued by the device, the gateway hub/smartphone app or both in order to alert the patient/user of faulty blood flow. Such an alarm or general feedback to the patient/user can e.g. be in the form of: sound, light, vibration, animation, graphs, or metrics. Some of these types of feedback require the information being given on a display whereas for others this is not necessary.

The system according to the invention can function both for long and short term monitoring. The presently intended use is from the time the patient undergoes AVF surgery until initiating haemodialysis treatment. This period normally takes 4-8 weeks, but it can take longer in case of complications. During this period, measurements are typically made every 30 minutes, but other intervals are also covered by the scope of protection. It could in principle be anything from continuous monitoring to e.g. once a day.

The system can be used in different ways including the following:
1) The device is constantly mounted on the patient and makes regular measurements at predetermined intervals for 4-8 weeks.
2) The device is not mounted to the patient, but instead it is placed at the location where the measurements are to be performed, such as the location of the AVF, e.g. twice a day to perform measurements.

During long term monitoring, the device can be mounted on the patient by use of reusable adhesive tape, which can be dried off with a wet cloth to restore the adhesive properties. Alternatively, the device can be mounted to the patient using a mesh bandage to secure the device in place during measurements. The mesh bandage must not compress the patient's arm as that might also compress e.g. an AVF and thereby reduce the blood flow so that the measurements become incorrect.

The individual aspects of the present invention may each be combined with any of the other aspects. These and other aspects of the invention will be apparent from the following description with reference to the described embodiments.

### BRIEF DESCRIPTION OF THE FIGURES

The device, the method and the system according to the invention will now be described in more detail with regard to the accompanying figures. The figures show one way of implementing the present invention and is not to be construed as being limiting to other possible embodiments falling within the scope of the attached claim set.

FIG. 1 schematically shows a device according to the invention.

FIG. 2 schematically shows an embodiment in which the device is a patch.

FIG. 3 schematically shows an embodiment of the invention in which the device further comprises a receiver and a memory.

FIG. 4 schematically shows an embodiment of a system according to the invention.

FIG. 5 is a flow diagram of an embodiment of a computer-implemented method according to the present invention.

FIG. 6 is a flow diagram of some embodiments of the method in FIG. 5.

FIG. 7 is a flow diagram of a part of a method according to an embodiment of the invention.

### DETAILED DESCRIPTION OF AN EMBODIMENT

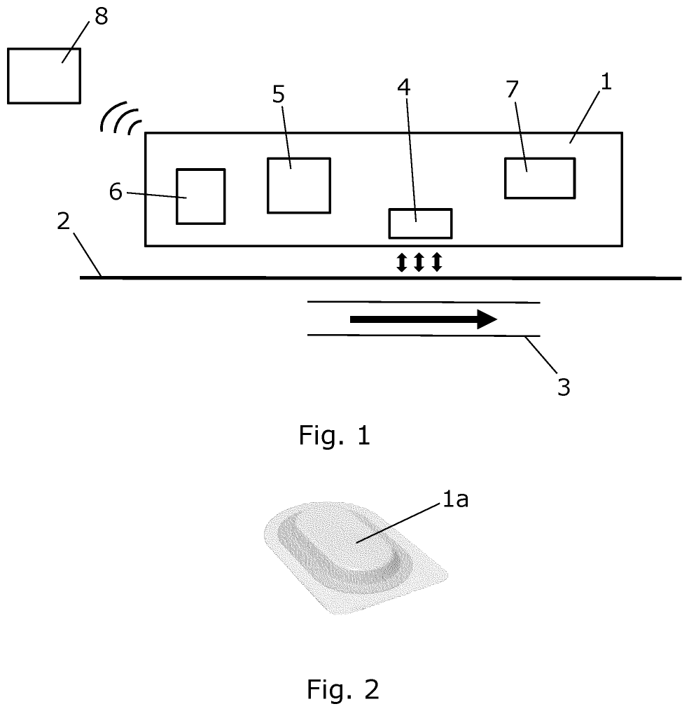

FIG. 1 schematically shows a device 1 according to the present invention. The device 1 is for sensing vibrations in a human's skin 2 emanating from blood flowing in a natural or artificial blood vessel or an arteriovenous fistula (AVF) 3 located below the skin 2. The way this is done will be described below. The device 1 comprises a sensor 4, a controller 5, a transmitter 6 and an electrical power source 7. The sensor 4 is configured to sense the vibrations when placed either directly or indirectly in contact with the human's skin 2 in close proximity to the natural or artificial blood vessel or AVF 3, and to provide a sensor readout representing said vibrations. The sensor 4 may e.g. be a photo plethysmography sensor, an accelerometer, or a sound sensor, such as a microphone.

The controller 5 is configured to sample the sensor readout and provide a sampled readout. The controller 5 may also be configured to perform predetermined processing of the sensor readout to arrive at the sampled readout.

The transmitter 6 is configured to transmit the sampled readout to an external processing unit 8. The transmitter 6 may e.g. be configured to transmit the sampled readout as a radio signal within in the ISM band from 2.402 GHz to 2.48 GHz.

The electrical power source 7 may e.g. be a battery, and it is used for supplying electrical power to the sensor 4, the controller 5, and the transmitter 6. The device 1 is configured to be fastened to the human and carried around by the human during use of the device 1. It may e.g. be in the form of a patch 1a carrying the sensor, the controller, the transmitter, and optionally the electrical power source.

FIG. 2 schematically shows an embodiment in which the device is a patch 1a. In general, one or more of the sensor 4, the controller 5, the transmitter 6, and the electrical power source 7 may be embedded in the patch 1a. In the illustrated embodiment, all of the components mentioned are embedded so that the patch 1a has a closed outer surface and so that the embedded components are not visible.

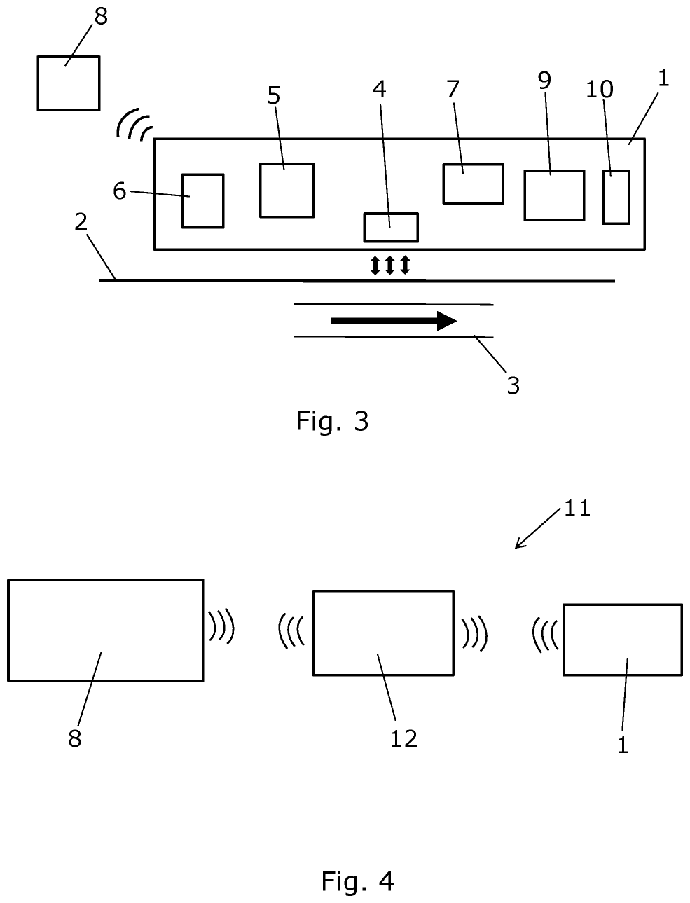

FIG. 3 schematically shows an embodiment of the invention in which the device 1 further comprises a receiver 9 configured to receive external input signals in response to which the controller 5 is controllable so that the sampling of sensor readout can be controlled via the external input signals. The embodiment in FIG. 3 further comprises a memory 10 for, at least temporarily, storing the sampled readout.

FIG. 4 schematically shows an embodiment of a system 11 for determining flow characteristics of a blood flow in natural or artificial a blood vessel or an arteriovenous fistula (AVF) located below a human's skin. The system 11 comprises a device 1 which may e.g. be in the form of a patch 1a, such as the one shown in FIG. 3. It further comprises a processing unit 8 configured to determine the flow characteristics by use of a method according to the invention. The system 11 further comprises a smartphone or a hub 12 via which the processing unit 8 can communicate with the device 1.

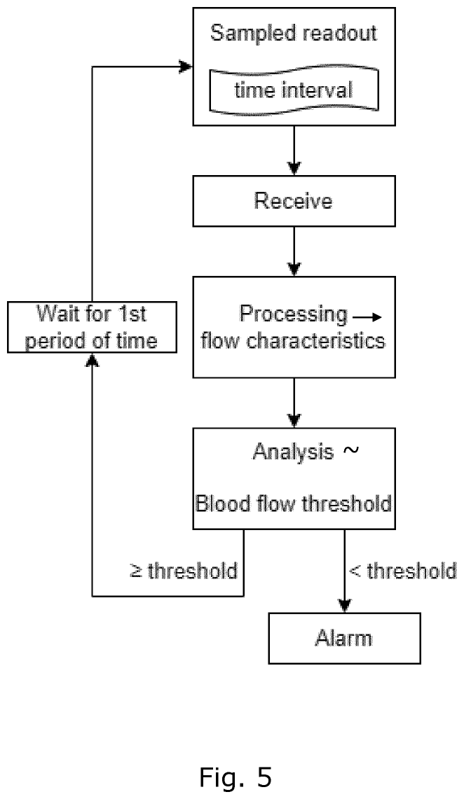

FIG. 5 is a flow diagram of an embodiment of a computer-implemented method according to the present invention. The method comprises the following steps:
providing, by use of the device 1, sampled readout spanning a pre-selected time interval,
receiving the sampled readout from the transmitter 6,
processing the sampled readout to determine corresponding flow characteristics of the blood flow in the natural or artificial blood vessel or AVF 3,
analysing the determined flow characteristics to establish whether or not the blood flow is at or above a blood flow threshold in the natural or artificial blood vessel or AVF 3, and
   if the blood flow is at or above the blood flow threshold, repeating the above steps after a pre-determined first period of time, and
   if the blood flow is below the blood flow threshold, issuing an alarm signal.

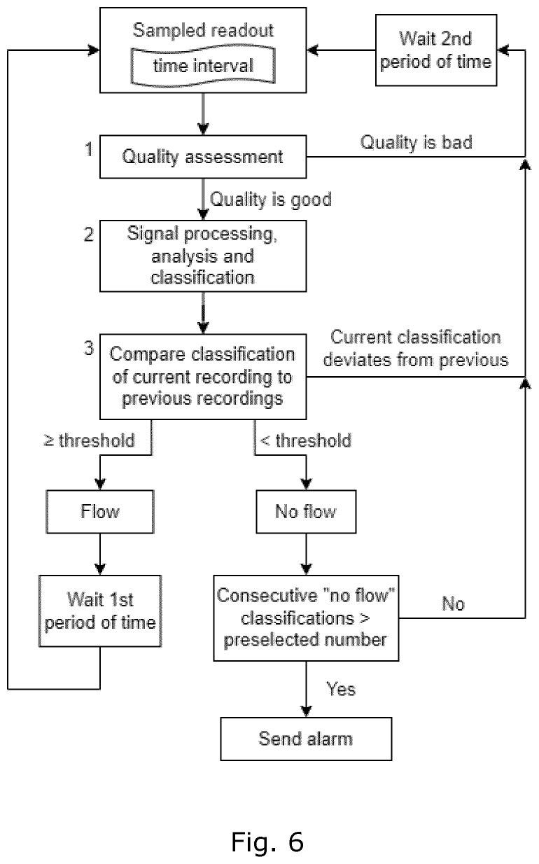

FIG. 6 is a flow diagram of some embodiments comprising more features than in the more general description shown in FIG. 5. In this embodiment, the step of processing the sampled readout comprises an initial step of assessing the quality of the received sampled readout, and:
if the sampled readout fulfils a pre-selected quality criteria, continue the processing, and
if the sampled readout does not fulfil the pre-selected quality criteria, discontinue the step of processing and wait for a pre-selected second period of time before repeating the step of processing on a subsequently sampled readout.

In FIG. 6, the fulfilment of the quality criteria is shown as “quality is good”, and non-fulfilment is shown as “quality is bad”. As described above, the step of assessing the quality may comprise determining how many times in a row, if any, it has been determined that the sampled readout does not fulfil the pre-selected quality criteria, and if this has happened more than a pre-selected number of times, the step of analysing is continued on the last sampled readout despite the non-fulfilment of the pre-selected quality criteria. This optional step is not included in FIG. 6.

The step of analysing the determined flow characteristics comprises:
assigning a classification to the determined flow characteristic,
comparing the assigned classification to the correspondingly assigned classification of at least one previously analysed flow characteristic, and
based thereon, establishing whether or not the blood flow in the natural or artificial blood vessel or AVF is at or above the blood flow threshold. In FIG. 6, it is shown that if the blood flow is determined to be at or above the blood flow threshold, it is considered to be “flow”, and if it is below the threshold, it is considered as “no flow”. This processing can be referred to as dichotomization, since data on a continuous scale are simplified to an “either-or-function”.

The embodiment in FIG. 6 further comprises determining how many times in a row, if any, it has been determined that the blood flow is below the blood flow threshold, and
when this has been determined a pre-selected number of times, issuing the alarm signal, and
when this has not been determined the pre-selected number of times, repeating the method steps after the pre-selected second period of time.

In some embodiments of the invention, machine learning is used to optimize the method. This may also include the use of deep-learning techniques. Utilizing machine learning can often ‘optimize’ the method or process of figuring out a functional way of differentiating between signals with and without flow. Since the machine learning model itself figures out how to classify signals, it can construct a more complex set of criteria for robust classification than what would be feasible for a human. The machine learning model is trained in a supervised manner. This means that it is provided with a large dataset of recordings, manually labelled into two classes; ‘flow’ or ‘no flow’. The model is then tasked to figure out characteristics for signals of each label. Provided that there is enough training data, the model should become robust at classifying new data.

In the following, an embodiment of the invention will be described in relation to the use for monitoring of an AVF; this embodiment includes the use of machine learning. A similar embodiment may be used for monitoring the blood flow in a natural or artificial blood vessel.

### Signal Analysis Algorithm Description

The algorithm used for analysing recordings made at the AVF by the device, overall has three main steps:
1) Assessment of the quality of the input signal
2) Signal processing, analysis and classification
3) Evaluation of classification results compared to previous results.
   In FIG. 6, the steps are marked by a number next to the box they represent. All signal processing in this embodiment is performed using MATLAB (R2021a), by The MathWorks Inc., Natick, Massachusetts. However, any software suitable for carrying out the invention is covered.

Following the flow diagram in FIG. 6 from top to bottom, the algorithm functions in the following steps:

First a new recording is made at the AVF by the device. This recording is 12 seconds long and sampled at 2000 Hz.

At step 1 the quality of the input signal is determined. This happens in two stages. In the first stage, the signal is first filtered by a band pass filter in frequency band 100 Hz to 300 Hz. Then the filtered signal is divided into sections of ⅔ of a second with a 50% overlap. This means that the 12 seconds signal is divided into 36sections. The first and last sections are removed to avoid zero-padding the signal for the remaining samples, resulting in 34 sections. For each section, the variance is calculated and the mean of all sections' variance is calculated. If a sections variance deviates more than 25% from the mean, that section is categorized as “bad”. If less than 23 (65%) of the sections are set as “bad”, then the whole signal is set as “bad” or zero, otherwise it is set as “good” or one. The reference to “zero” and “one” refers to this embodiment where a binary categorization is used.

In the next stage, the unfiltered 12 seconds signal is divided into 72 sections of ⅓ of a second also with 50% overlap; again the first and last sections are removed to avoid zero-padding the signal for the remaining samples, resulting in 70 sections. This is to capture abrupt changes in the signal. For each section, the minimum and maximum values are found and the mean of the two is calculated.

Then the mean of all sections' mean values is calculated. If a section's minimum/maximum mean deviates more than 10% from the overall mean, then that section is set as “bad”. If less than 18 (25%) of the sections are set as “bad” then the whole signal is set as “bad” or zero, otherwise it is set as “good” or one.

Finally, an OR-gate is used to decide the quality of the input signal. This means that if both or either of the two quality checks has set a signal as “good”, the quality of the signal is decided as “good”. If at both stages, the quality checks have set the signal as “bad”, then the signal quality is decided as “bad” and the signal is discarded and a new recording is made after 10 minutes. If three consecutive recordings have been labelled as “bad” quality, the fourth is forced through to avoid the algorithm getting caught in an infinite loop where no recording is ever analysed.

Otherwise, if the signal quality is good, the algorithm moves to step 2 where the signal is pre-processed, analysed and classified. Step two is described in greater detail later in FIG. 6.

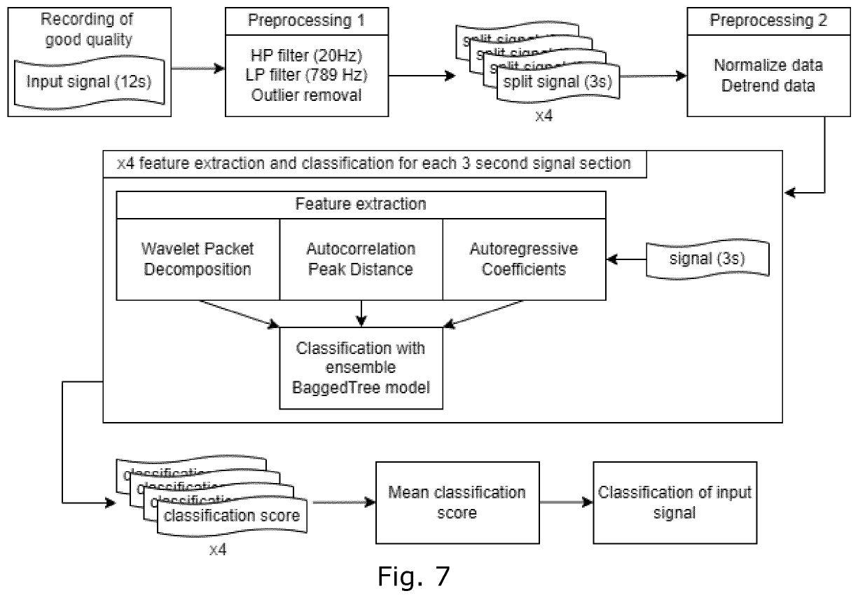

In step 2 a recorded signal of good quality is the input. Details of this step 2 is shown in FIG. 7. First the signal is pre-processed by filtering with a 4^{th} order Butterworth high pass filter with cut-off frequency at 20 Hz. This is to filter off movement artefacts produced by the patient. Next it is filtered by a 4^{th} order Butterworth low pass filter with cut-off frequency at 789 Hz. This is because of a hardware limitation with the currently used accelerometer (ADXL355). Next outliers are removed using the built-in MATLAB function ‘filloutliers.m’.

Then the 12 seconds signal is split into four sections each of 3 seconds length. Each of the four signal sections are processed through the following steps. The signal is normalized to a range between −1 and 1. Then it is detrended using the built-in MATLAB function ‘detrend.m’ to remove possible linear trends in the data. Three different features are then calculated for the signal; energy percentage, autocorrelation peak distance and autoregressive coefficients.

Energy Percentage is calculated by Wavelet Packet Decomposition (WPD). The energy percentage is the percentage of total energy within specific frequency bands of a wavelet package decomposition. The WPD decompose the signal at level 4 using wavelet Daubechies 4. This produces a “wavelet packet tree”, with terminal nodes accounting for a frequency band of the input signals sample rate (0-2000 Hz). At level 4 each band is 125 Hz wide. Only the nodes 1, 2 and 6 are used corresponding to frequency bands: 0-125 Hz, 125-250 Hz and 625-750 Hz. The logarithm of the energy in the three bands are calculated and the results are the features used.

Autocorrelation peak distance is calculated by creating an envelope of the signal by rectifying and low pass filtering with a 4^{th} order Butterworth filter with cut-off frequency at 2.5 Hz. The peaks are then found moving forward from lag zero of the autocorrelation. The distance from lag zero to the first peak is used as feature.

Autoregressive Coefficients are found by calculating an autoregressive model of 4^{th} order using Burg's Method with the built-in MATLAB function ‘arburg.m’. Only the second autoregressive parameter returned by the model is used as a feature. The result is a total of five features: 3 energy percentage, 1 autocorrelation peak distance and 1 autoregressive. The five features are passed to a pre-trained bootstrap-aggregated decision tree (BaggedTree) classification ensemble trained with the built-in MATLAB function ‘fitensemble.m’. The classification model classifies based on a classification score where the highest score decides one of the two classes; “flow” and “no flow”.

The mean of the four 3 seconds section classification scores is calculated and decides the overall classification for the 12 seconds input signal. The classification is passed on to step 3 as described above.

In step 3 the classification scores of the classification in the step 2 are compared. When a new recording has been classified, the score of that recording is compared to the classification scores of the previous three recordings. It is decided if a classification score deviates too much by calculating the mean of the previous three scores and finding the standard deviation. If the current score is deviating more than the standard deviation from the mean, then it is deviating too much from previous scores and is “not trusted”. Additionally, scores over 90% certainty are automatically trusted.

Lastly, the classification of a recording, either “flow” or “no flow”, decides if the algorithm should either continue by waiting 30 minutes and making a new recording and start over or check if the current recording is the third recording in a row labelled and trusted as “no flow”, then an alarm is sent. If the current classification is labelled and trusted as “no flow”, but is not the third in a row, the algorithm will wait 10 minutes and then make a new recording. This is to ensure that the algorithm does not send an alarm in case of e.g. only one false positive classification.

In addition to the analyses being described above, the algorithm may be configured to determine one or more of the following parameters based on the measurements:
The flow rate (ml/min) in the natural or artificial blood vessel or AVF.
Changes in the flow rate based on comparisons with previous measurements.
The maturation of an AVF to help determine when it is ready for use in haemodialysis treatment.
The heart rate of the patient carrying the device.

The maturation of an AVF has typically occurred when the diameter of the AVF is at least 4 mm to 5 mm in diameter, and it has a flow rate of at least 500 mL/min. However, the vein may need to be between 6 mm to 7 mm in diameter or greater, before the average technologist or nurse at a dialysis centre can reliably perform repeated cannulation of the AVF without infiltrating or damaging it. In prior art, the maturation is typically detected by use if ultrasound analysis, but with the present invention, it may be possible to extract this information directly from the measurement data.

A device or system according to the invention may furthermore be provided with one or more of the following functionalities:
A function to enable being found if lost. This can e.g. be obtained by configuring the device to be able to give off sound, light, or vibration by activating a “find” functionality in the gateway hub/smartphone app.
An “out of range” alarm so that when the device gets too far away from the gateway hub/smartphone app, or gets a bad connection or lose connection to the gateway hub/smartphone app, it will issue an alarm. Hereby the user can take appropriate action e.g. by moving closer to the gateway hub/smartphone.
It can be configured to notify the patient that a measurement is being made so that the patient/user knows to remain reasonably still.
   It can be configured to notify the patient/user to move his or her arm, e.g. in case the patient/user is laying on the arm causing reduced or no blood flow in the AVF, e.g. during sleep.
   The invention can be implemented by means of hardware, software, firmware or any combination of these. The invention or some of the features thereof can also be implemented as software running on one or more data processors and/or digital signal processors.

The individual elements of an embodiment of the invention may be physically, functionally and logically implemented in any suitable way such as in a single unit, in a plurality of units or as part of separate functional units. The invention may be implemented in a single unit, or be both physically and functionally distributed between different units and processors.

Although the present invention has been described in connection with the specified embodiments, it should not be construed as being in any way limited to the presented examples. The scope of the present invention is to be interpreted in the light of the accompanying claim set. In the context of the claims, the terms “comprising” or “comprises” do not exclude other possible elements or steps. Also, the mentioning of references such as “a” or “an” etc. should not be construed as excluding a plurality. The use of reference signs in the claims with respect to elements indicated in the figures shall also not be construed as limiting the scope of the invention. Furthermore, individual features mentioned in different claims, may possibly be advantageously combined, and the mentioning of these features in different claims does not exclude that a combination of features is not possible and advantageous.

### List of Reference Symbols Used

1 Device
1a Patch
2 Skin
3 Blood vessel or AVF
4 Sensor
5 Controller
6 Transmitter
7 Power source
8 Processing unit
9 Receiver
10 Memory
11 System
12 Smartphone or hub

1. A device for sensing vibrations in a human's skin emanating from blood flowing in a natural or artificial blood vessel or an arteriovenous fistula (AVF) located below the skin, the device comprising:
a sensor configured to:
sense the vibrations when placed either directly or indirectly in contact with the human's skin in proximity to the natural or artificial blood vessel or AVF, and
provide a sensor readout representing said vibrations;
a controller configured to sample the sensor readout and provide a sampled readout,
a transmitter configured to transmit the sampled readout to an external processing unit, and
an electrical power source for supplying electrical power to the sensor, controller, and transmitter,
wherein the device is configured to be fastened to the human and carried around by the human during use of the device.

2. A device according to claim 1, wherein the sensor is a photo plethysmography sensor, an accelerometer, or a sound sensor.

3. A device according to claim 1, wherein the device comprises a patch that includes the sensor, the controller, the transmitter, and optionally includes the electrical power source.

4. A device according to claim 3, wherein any one or more of the sensor, the controller, the transmitter, and or the electrical power source is embedded in the patch.

5. A device according to claim 1, wherein the transmitter is configured to transmit the sampled readout as a radio signal within in the ISM band from 2.402 GHz to 2.48 GHz.

6. A device according to claim 1, further comprising a receiver configured to receive external input signals in response to which the controller is controllable so that the sampling of sensor readout can be controlled via the external input signals.

7. A device according to claim 1, further comprising a memory for storing the sampled readout.

8. A device according to claim 1, wherein the controller is configured to perform predetermined processing of the sensor readout to arrive at the sampled readout.

9. A device according to claim 1, wherein the device is further provided with any one or more of the following hardware components: an internal real-time clock (RTC), a contact microphone, or a dynamic speaker enabling healthcare personnel to speak directly to user of the device.

10. A device according to claim 1, the device being configured to be in a state of one among several modes of operation.

11. A computer-implemented method for determining flow characteristics of a blood flow in a natural or artificial blood vessel or an arteriovenous fistula (AVF) below a human's skin, the method comprising:
providing, by use of a device, sampled readout spanning a pre-selected time interval,
receiving the sampled readout from a transmitter of the device,
processing the sampled readout to determine corresponding flow characteristics of the blood flow in the natural or artificial blood vessel or AVF,
analysing the determined flow characteristics to establish whether or not the blood flow is at or above a blood flow threshold in the natural or artificial blood vessel or AVF, and
if the blood flow is at or above the blood flow threshold, repeating the providing, receiving, processing, and analysing after a pre-determined first period of time, and
if the blood flow is below the blood flow threshold, issuing an alarm signal.

12. A computer implemented method according to claim 11, wherein the step of processing the sampled readout comprises an initial step of assessing a quality of the received sampled readout, and:
if the sampled readout fulfils a pre-selected quality criteria, continue the processing, and
if the sampled readout does not fulfil the pre-selected quality criteria, discontinue the step of processing and wait for a pre-selected second period of time before repeating the step of processing on a subsequently sampled readout.

13. A computer-implemented method according to claim 12, wherein the step of assessing the quality comprises determining how many times in a row, if any, it has been determined that the sampled readout does not fulfil the pre-selected quality criteria, and if this has happened more than a pre-selected number of times, the step of analysing is continued on the last sampled readout despite the non-fulfilment of the pre-selected quality criteria.

14. A computer-implemented method according to claim 11, wherein the step of analysing the determined flow characteristics comprises:
assigning a classification to the determined flow characteristic,
comparing the assigned classification to the correspondingly assigned classification of at least one previously analysed flow characteristic, and
based thereon, establishing whether or not the blood flow in the natural or artificial blood vessel or AVF is at or above the blood flow threshold.

15. A computer-implemented method according to claim 14, further comprising determining how many times in a row, if any, it has been determined that the blood flow is below the blood flow threshold, and
when the blood flow is below the blood flow threshold has been determined a pre-selected number of times, issuing the alarm signal, and
when the blood flow is below the blood flow threshold has not been determined the pre-selected number of times, repeating the assigning, comparing, and establishing after a pre-selected second period of time.

16. A system for determining flow characteristics of a blood flow in natural or artificial a blood vessel or an arteriovenous fistula (AVF) located below a human's skin, the system comprising:
a device according to claim 1, and
a processing unit configured to perform at least:
providing, by use of the device, sampled readout spanning a pre-selected time interval,
receiving the sampled readout from the transmitter,
processing the sampled readout to determine corresponding flow characteristics of the blood flow in the natural or artificial blood vessel or AVF,
analysing the determined flow characteristics to establish whether or not the blood flow is at or above a blood flow threshold in the natural or artificial blood vessel or AVF,
in order to determine the flow characteristics.

17. A system according to claim 16, wherein the system further comprises a smartphone or a hub via which the processing unit can communicate with the device.

18. A system according to claim 17, wherein the processing unit is a cloud server where data is analysed and stored.

19. A device according to claim 10, wherein a mode of operation is any one or more of (1) shelf/low-power, (2) sleep, (3) awake, (4) boot, (5) connecting, (6) measurement, (7) transmit, (8) command, or (9) debug.

20. A system according to claim 16, wherein the processing unit is configured such that
if the blood flow is at or above the blood flow threshold, the system repeats the providing, receiving, processing, and analysing after a pre-determined first period of time, and
if the blood flow is below the blood flow threshold, the system issues an alarm signal.
